(19) United States
(12) Patent Application Publication
(10) Pub. No.: US 2025/0345513 A1
(43) Pub. Date: Nov. 13, 2025

(54) FLUID MANAGEMENT SYSTEM WITH INTEGRAL PRESSURE REGULATION MECHANISM

(71) Applicant: Stryker Corporation, Portage, MI (US)

(72) Inventor: Benjamin Theodore LORMAN, Solon, OH (US)

(73) Assignee: Stryker Corporation, Portage, MI (US)

(21) Appl. No.: 19/201,644

(22) Filed: May 7, 2025

Related U.S. Application Data
(60) Provisional application No. 63/644,093, filed on May 8, 2024.

(51) Int. Cl. A61M 5/142; A61M 1/00; A61M 5/00; A61M 5/168

(52) U.S. Cl. CPC A61M 5/14228; A61M 1/74; A61M 5/16809; A61M 5/007; A61M 2205/3327; A61M 2205/3365

(57) ABSTRACT

A fluid management system for medical usage comprises: a first chamber having a fluid inlet; a second chamber having a fluid outlet; a third chamber configured to be pressurized at a pressure setpoint, wherein the third chamber is connected to the first chamber via a first set of one or more openings and connected to the second chamber via a second set of one or more openings; a pump configured to transfer fluid received from the first chamber to the second chamber; and a flexible membrane fluidly isolating the third chamber from both the first chamber and the second chamber, wherein the flexible membrane is movable by a differential pressure between the third chamber and the second chamber such that the first chamber and the second chamber are fluidly connected via the first set of one or more openings and the second set of one or more openings.

### CROSS-REFERENCE TO RELATED APPLICATION

This application claims the benefit of U.S. Provisional Application 63/644,093 filed on May 8, 2024, the entire content of which is incorporated herein by reference for all purposes.

### FIELD

The present disclosure relates generally to fluid management systems, and more specifically to fluid management systems with pressure regulation mechanisms.

### BACKGROUND

In medical procedures, various types of pumps are used to deliver fluids (e.g., saline solution, medications, contrast agents) to and/or remove fluids (e.g., blood, secretions, debris) from target sites. For example, peristaltic pumps are commonly used in endoscopy procedures to deliver fluids, such as saline solution or contrast agents, into the gastrointestinal tract or other areas being examined. Importantly, different organs or anatomical structures may be associated with different acceptable pressure thresholds depending on factors such as tissue sensitivity, vascularization, and the type of procedure being performed. For example, the pressure at which fluids can be delivered to a bladder should generally not exceed 75 millimeters of mercury. However, without proper pressure regulation, a pump may deliver fluids at an excessive pressure (e.g., due to high pump speed, obstruction at the target site, etc.), thus compromising patient safety. Thus, it is important to regulate fluid pressure promptly during medical procedures.

However, the pressure regulation mechanisms of many existing pumps have several limitations. Backpressure regulators are typically not cost-effective enough to be deployed at scale in single-use sterile medical devices as would be required for irrigation pumps and transfusion pumps. Therefore, the primary pressure regulation mechanism is currently achieved by regulating the speed of the peristaltic pump using feedback from a pressure transducer. Due to the delay caused by processing feedback from the pressure sensors and the timing of various electronics, pressure regulation at many existing pumps is not responsive and a pump can take a relatively long time to achieve a desirable pressure setpoint. Furthermore, many existing pressure regulation mechanisms may fail to maintain sterile integrity of the fluid. For example, some medical pumps contain pressure relief regulators in disposable tubing, but they are only used as a last resort because they are designed to vent fluid (e.g., spraying liquid onto the floor) beyond a fixed pressure setpoint, thus potentially compromising the sterile integrity of the fluid and contaminating the surgical environment.

### SUMMARY

Disclosed herein are fluid management systems with integral pressure regulation mechanisms for medical usage. The fluid management systems disclosed herein can be configured to deliver fluids (e.g., saline solution, medications, contrast agents) to and/or remove fluids (e.g., blood, secretions, debris) from target sites. An exemplary fluid management system comprises a first chamber having a fluid inlet and a second chamber having a fluid outlet. A pump (e.g., a peristaltic pump) is configured to transfer fluid received from the first chamber to the second chamber. A third chamber is connected to the first chamber via a first set of one or more openings and connected to the second chamber via a second set of one or more openings and is configured to be pressurized at a pressure setpoint. To regulate pressure of the fluid at the fluid outlet (which is the same as the pressure within the second chamber), the fluid management system comprises a flexible membrane that fluidly isolates the third chamber from both the first chamber and the second chamber. The flexible membrane is movable by a differential pressure between the third chamber and the second chamber such that the first chamber and the second chamber can be fluidly connected via the first set of one or more openings and the second set of one or more openings.

In an exemplary configuration, when the pressure in the third chamber (i.e., the pressure setpoint) equals or is higher than the pressure in the second chamber, the pressure in the third chamber pushes the flexible membrane to seal off the first set of openings and the second set of openings. Thus, the fluid delivered at the fluid outlet has the same pressure as the pressure in the second chamber, which equals or is lower than the pressure in the third chamber (i.e., the pressure setpoint). On the other hand, when the pressure in the third chamber (i.e., the pressure setpoint) is lower than the pressure in the second chamber, the higher pressure in the second chamber displaces the flexible membrane to open a fluid passage for fluid to travel through the second set of openings into the third chamber and then through the first set of openings into the first chamber. The circulation of fluid from the second chamber back to the first chamber reduces the pressure in the second chamber. Accordingly, the fluid delivered at the fluid outlet is generally delivered at a lower or identical pressure as the pressure setpoint.

The fluid management systems described herein provide several technical advantages. The configuration automatically relieves pressure via mechanical means and thus eliminates the need for measuring the pressure of the fluid using pressure sensors under constantly fluctuating fluid path conditions, analyzing the pressure readings, and relying on electronics to regulate fluid pressure. Thus, a faster response time can be achieved, which is particularly beneficial for medical procedures. Further, the pressure regulation mechanism redirects any fluid released during pressure relief to the inlet of the pump rather than releasing the fluid into the surgical environment (e.g., spilling it on the floor), thus maintaining the sterile integrity of the fluid and preventing wastage of fluid.

Further, provided that the openings in the fluid management system are sized to vent the maximum flow rate capable of being supplied by the pump, consistent pressure regulation is achieved while allowing the pump to run consistently at the maximum speed. Under the constantly changing conditions of the fluid path during certain medical procedures (e.g., endoscopic surgeries), flow to the surgical site is automatically adjusted while maintaining constant pressure. The pressure setpoint of the regulation mechanism guarantees the fluid pressure delivered to the surgical site. Lastly, the design of the pressure regulation mechanism is cost-effective enough for inclusion in a single-use disposable medical device.

A fluid management system for medical usage comprises: a first chamber having a fluid inlet; a second chamber having a fluid outlet; a third chamber configured to be pressurized at a pressure setpoint, wherein the third chamber is connected to the first chamber via a first set of one or more openings and connected to the second chamber via a second set of one or more openings; a pump configured to transfer fluid received from the first chamber to the second chamber; and a flexible membrane fluidly isolating the third chamber from both the first chamber and the second chamber, wherein the flexible membrane is movable by a differential pressure between the third chamber and the second chamber such that the first chamber and the second chamber are fluidly connected via the first set of one or more openings and the second set of one or more openings.

In some examples, the fluid transferred by the pump comprises liquid and/or air, and the fluid management system is configured to receive the fluid from the fluid source and provide the fluid via the fluid outlet at the pressure setpoint.

In some examples, the third chamber is configured to be pressurized via air.

In some examples, the liquid comprises saline, a contrast agent, a medication, or any combination thereof. In some examples, the fluid source comprises one or more intravenous (IV) containers and the fluid outlet is connected to a patient.

In some examples, the fluid transferred by the pump comprises liquid and/or air, and the fluid management system is configured to receive the fluid from a surgical site and provide the fluid via the fluid outlet at the pressure setpoint.

In some examples, the third chamber is a vacuum chamber.

In some examples, the fluid outlet is connected to a waste drain.

In some examples, the pump comprises a peristaltic pump.

In some examples, the pressure setpoint in the third chamber is specified by a user input. In some examples, the user input is received by a software-controlled pump configured to regulate pressure in the third chamber.

In some examples, the third chamber comprises an intermediate chamber adjacent to the first chamber and the second chamber, and the flexible membrane is located within the intermediate chamber.

In some examples, the fluid management system comprises a sensor for detecting displacement of the flexible membrane. In some examples, the sensor comprises a motion sensor.

In some examples, the fluid management system is configured to reduce a speed of the pump if the displacement of the flexible membrane is detected.

In some examples, the fluid management system is configured to stop the pump if the displacement of the flexible membrane exceeds a threshold time period.

An exemplary method for providing fluid management comprises: receiving fluid at a first chamber of a fluid management system via a fluid inlet of the first chamber; transferring the fluid from the first chamber to a second chamber of the fluid management system via a pump; pressuring a third chamber of the fluid management system at a pressure setpoint, wherein the third chamber is connected to the first chamber via a first set of one or more openings and connected to the second chamber via a second set of one or more openings; moving, via a differential pressure between the second chamber and the third chamber, a flexible membrane fluidly isolating the third chamber from both the first chamber and the second chamber, such that the first chamber and the second chamber are fluidly connected via the first set of one or more openings and the second set of one or more openings; and releasing the fluid from the second chamber via a fluid outlet of the second chamber.

In some examples, the fluid transferred by the pump comprises liquid and/or air, and the fluid management system is configured to receive the fluid from the fluid source and provide the fluid via the fluid outlet at the pressure setpoint.

An exemplary fluid management system configured to be coupled to a pump for medical usage comprises: a first chamber having a fluid inlet, wherein the first chamber is configured to obtain fluid via the fluid inlet; a second chamber having a fluid outlet, wherein the second chamber is configured to receive the fluid via the pump and release the fluid via the fluid outlet; a third chamber configured to be pressurized at a pressure setpoint, wherein the third chamber is connected to the first chamber via a first set of one or more openings and connected to the second chamber via a second set of one or more openings; and a flexible membrane fluidly isolating the third chamber from both the first chamber and the second chamber, wherein the flexible membrane is movable by a differential pressure between the third chamber and the second chamber such that the first chamber and the second chamber are fluidly connected via the first set of one or more openings and the second set of one or more openings.

In some examples, the fluid transferred by the pump comprises liquid and/or air, and wherein the fluid management system is configured to receive the fluid from the fluid source and provide the fluid via the fluid outlet at the pressure setpoint.

### BRIEF DESCRIPTION OF THE FIGURES

FIGS. 1A-1B illustrate an exemplary surgical environment, in accordance with some examples.

FIGS. 1C-1E illustrate exemplary operations of a peristaltic pump, in accordance with some examples.

FIGS. 2A-2B illustrates an exemplary fluid management system for medical usage, in accordance with some examples.

FIGS. 3A-3B illustrate an exemplary fluid management system for medical usage, in accordance with some examples.

FIG. 4 illustrates an exemplary fluid management system for medical usage, in accordance with some examples.

FIG. 5 illustrates an exemplary method for providing fluid management, in accordance with some examples.

FIG. 6 illustrates an exemplary computer system, in accordance with some examples.

FIGS. 7A-7D illustrate an exemplary fluid management system for medical usage, in accordance with some examples.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Disclosed herein are fluid management systems with integral pressure regulation mechanisms for medical usage. The fluid management systems disclosed herein can be configured to deliver fluids (e.g., saline solution, medications, contrast agents) to and/or remove fluids (e.g., blood, secretions, debris) from target sites. An exemplary fluid management system comprises a first chamber having a fluid inlet and a second chamber having a fluid outlet. A pump (e.g., a peristaltic pump) is configured to transfer fluid received from the first chamber to the second chamber. A third chamber is connected to the first chamber via a first set of one or more openings and connected to the second chamber via a second set of one or more openings and is configured to be pressurized at a pressure setpoint. To regulate pressure of the fluid at the fluid outlet (which is the same as the pressure within the second chamber), the fluid management system comprises a flexible membrane that fluidly isolates the third chamber from both the first chamber and the second chamber. The flexible membrane is movable by a differential pressure between the third chamber and the second chamber such that the first chamber and the second chamber can be fluidly connected via the first set of one or more openings and the second set of one or more openings.

In an exemplary configuration, when the pressure in the third chamber (i.e., the pressure setpoint) equals or is higher than the pressure in the second chamber, the pressure in the third chamber pushes the flexible membrane to seal off the first set of openings and the second set of openings. Thus, the fluid delivered at the fluid outlet has the same pressure as the pressure in the second chamber, which equals or is lower than the pressure in the third chamber (i.e., the pressure setpoint). On the other hand, when the pressure in the third chamber (i.e., the pressure setpoint) is lower than the pressure in the second chamber, the higher pressure in the second chamber displaces the flexible membrane to open a fluid passage for fluid to travel through the second set of openings into the third chamber and then through the first set of openings into the first chamber. The circulation of fluid from the second chamber back to the first chamber reduces the pressure in the second chamber. Accordingly, the fluid delivered at the fluid outlet is generally delivered at a lower or identical pressure as the pressure setpoint.

The fluid management systems described herein provide several technical advantages. The configuration automatically relieves pressure via mechanical means and thus eliminates the need for measuring the pressure of the fluid using pressure sensors under constantly fluctuating fluid path conditions, analyzing the pressure readings, and relying on electronics to regulate fluid pressure. Thus, a faster response time can be achieved, which is particularly beneficial for medical procedures. Further, the pressure regulation mechanism redirects any fluid released during pressure relief to the inlet of the pump rather than releasing the fluid into the surgical environment (e.g., spilling it on the floor), thus maintaining the sterile integrity of the fluid and preventing wastage of fluid.

Further, provided that the openings in the fluid management system are sized to vent the maximum flow rate capable of being supplied by the pump, consistent pressure regulation is achieved while allowing the pump to run consistently at the maximum speed. Under the constantly changing conditions of the fluid path during certain medical procedures (e.g., endoscopic surgeries), flow to the surgical site is automatically adjusted while maintaining constant pressure. The pressure setpoint of the regulation mechanism guarantees the fluid pressure delivered to the surgical site. Lastly, the design of the pressure regulation mechanism is cost-effective enough for inclusion in a single-use disposable medical device.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating,” or the like refer to the action and processes of a computer system, or similar electronic computing device, that manipulates and transforms data represented as physical (electronic) quantities within the computer system memories or registers or other such information storage, transmission, or display devices.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

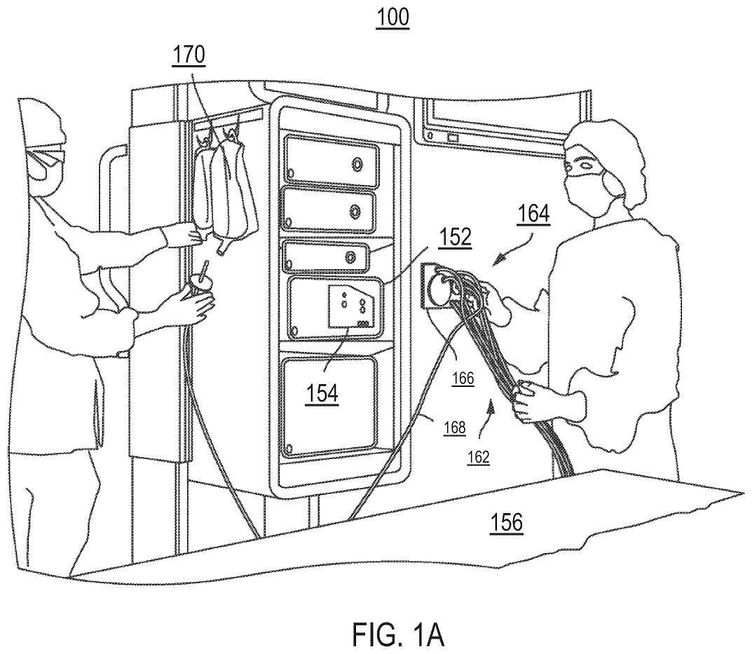

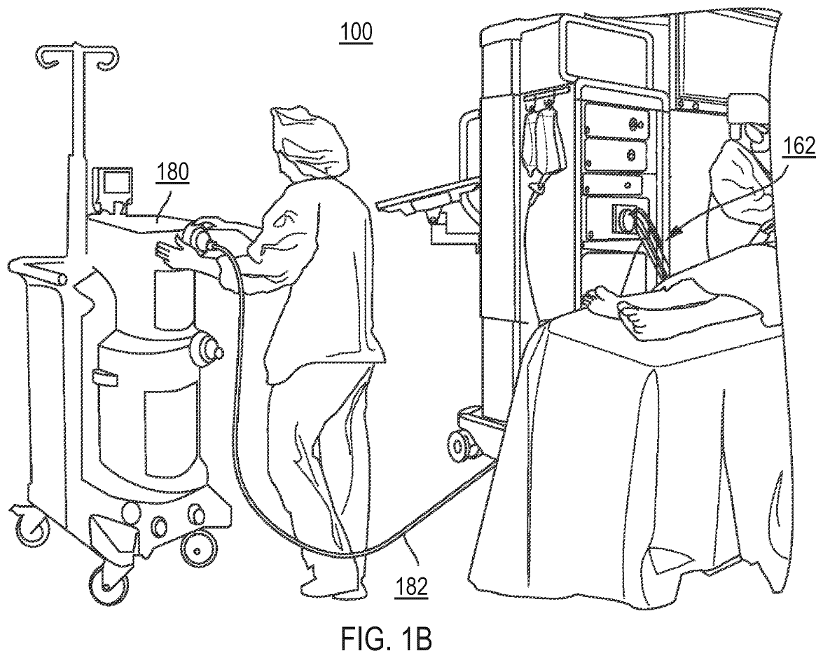

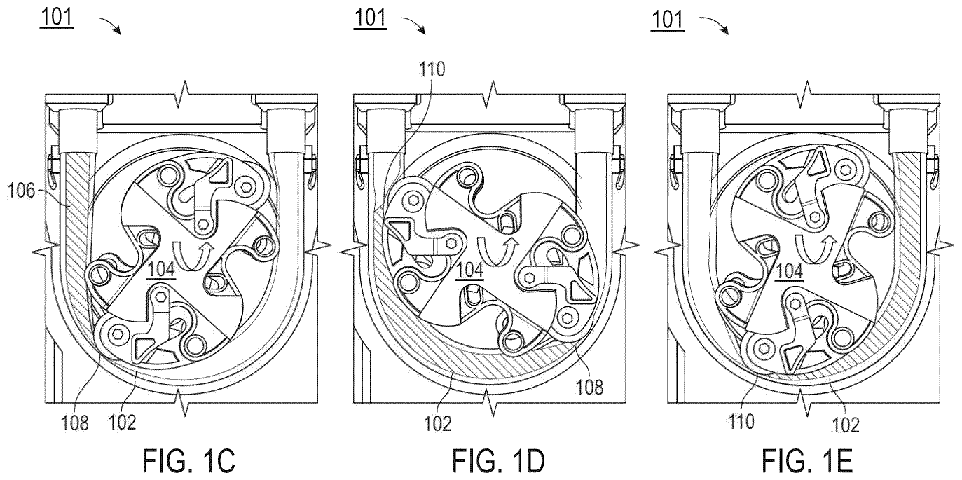

FIGS. 1A-B illustrate an exemplary surgical environment 100 (e.g., an operating room), in accordance with some examples. With reference to FIGS. 1A-B, the surgical environment 100 can include a fluid supply management system 152 that can operate according to the techniques described herein. The fluid supply management system 152 can be located near a surgical table 156 to be within reach of the surgical staff to deliver fluids to and/or remove fluids from patients during surgical procedures.

In the depicted example in FIGS. 1A-B, a tube set 162 is connected to the fluid supply management system 152. The non-patient end 164 of the tube set 162 includes a connector 166 to which some or all of the tubes of the tube set 162 are pre-attached. The connector 166 is connected to the receptacle 154 of the fluid supply management system 152. In the illustrated example, the tube set 162 includes a liquid supply tube 168 that is connected to a liquid supply reservoir 170, which can be for example a saline bag, for supplying liquid to the surgical target site, such as for the scope cleaner and/or irrigation supply device. FIG. 1B illustrates the connection of a suction line 182 of the tube set 162 to a waste collection apparatus 180. Although not shown, one or more lines in the tube set 162 can be connected to other equipment in the operating room.

FIGS. 1C-E illustrate exemplary operations of a peristaltic pump 101, in accordance with some examples. The peristaltic pump 101 moves fluids by repeatedly enclosing a fixed volume of fluids and moving the fixed volume mechanically through the system. With reference to FIG. 1C, the peristaltic pump 101 comprises a flexible tube 102. The flexible tube 102 is made of a flexible material (e.g., silicone, rubber, or the like) that can withstand repeated compression and expansion. The peristaltic pump 101 further comprises a set of rollers 104 that can be configured to rotate to compress and release the flexible tube 102 to move fluids through the flexible tube 102.

In operation, by compressing and releasing the flexible tube 102 in a rhythmic manner, the set of rollers 104 sequentially compress sections of the flexible tube 102 against a fixed surface, thereby creating a series of fixed volumes of fluid and pushing the fluid forward in the flexible tube 102. In FIG. 1C, the set of rollers 104 compresses the flexible tube 102 to create a first point of compression 108. As the set of rollers 104 rotates further (e.g., counterclockwise as shown in FIGS. 1C-E), the flexible tube 102 draws in fluid 106. As the set of rollers 104 continues to rotate while maintaining the first point of compression 108 on the flexible tube 102, more fluid is drawn into the flexible tube 102. In FIG. 1D, the set of rollers 104 is further rotated such that it compresses the flexible tube 102 at both the first point of compression 108 and a second point of compression 110, thus enclosing a fixed volume of fluid and moving the fixed volume of fluid through the flexible tube 102. In FIG. 1E, the set of rollers 104 is further rotated such that it releases the first point of compression 108 (depicted in FIG. 1D and the flexible tube 102 returns to its original shape. The rotation further moves the fixed volume of fluid out of the flexible tube 102 (not shown in figure). Accordingly, the repeated compression and release of the flexible tube 102 generate a continuous flow of fluid through the pump 101.

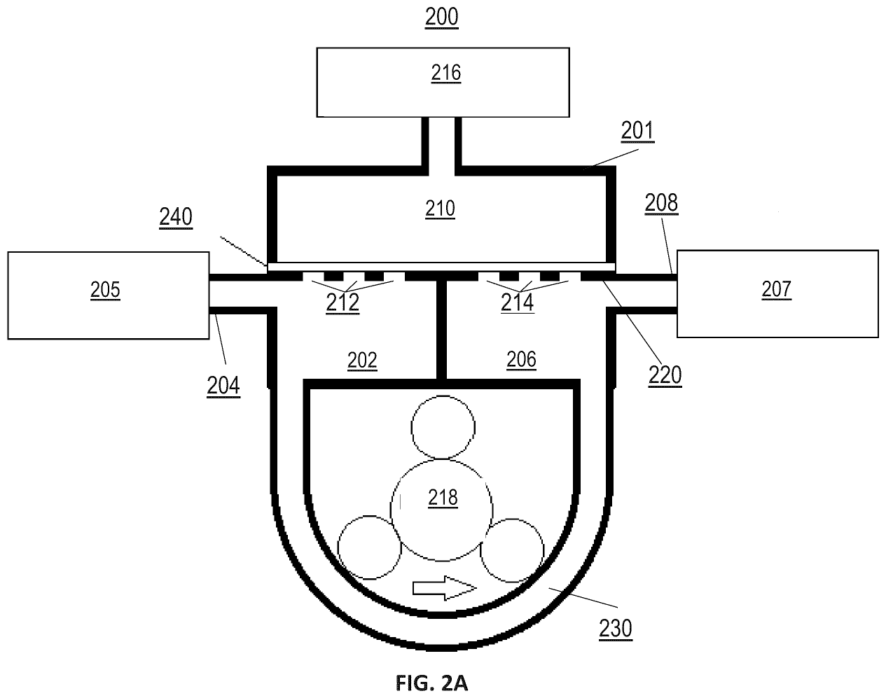

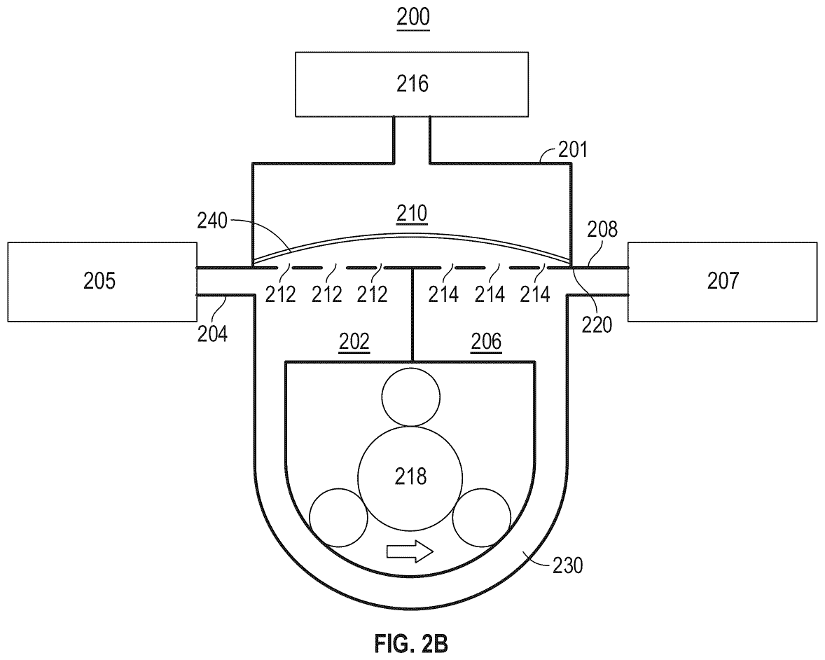

FIGS. 2A-B illustrate an exemplary fluid management system 200 for medical usage, in accordance with some examples. The fluid management system 200 comprises a body 201 (e.g., a disposable cassette), which can be coupled to a fluid source 205, a delivery site 207, a pressure regulation device 216 (e.g., a software-controlled pump), and a pump 218. In some examples, the body 201 can be made of a disposable material. Accordingly, the body 201 can be detached from the other components and discarded after use (e.g., after a medical procedure) and the other components can be re-used with a new body (e.g., to manage a different type of fluid, to be used for a different patient and/or in a different medical procedure).

With reference to FIGS. 2A-B, the fluid management system 200 (specifically, the body 201) comprises a first chamber 202 having a fluid inlet 204 and a second chamber 206 having a fluid outlet 208. The fluid management system 200 further comprises a pump 218. The pump 218 is configured to transfer fluid received from the first chamber 202 to the second chamber 206. In some examples, the pump 218 is a peristaltic pump that can operate as described with reference to FIGS. 1A-C to draw fluid into the first chamber 202 via the fluid inlet 204, move the fluid through a flexible tube 230 to the second chamber by compressing and releasing the flexible tube 230 via a set of rollers, and extrude the fluid out of the second chamber 206 via the fluid outlet 208.

In the depicted example in FIGS. 2A-B, the fluid inlet 204 can be connected to a fluid source 205. The fluid source 205 can provide a variety of fluids, such as liquid (e.g., saline, a contrast agent, a medication, or the like) or air, to the fluid management system 200. In some examples, the fluid source 205 comprises one or more intravenous (IV) containers. The fluid outlet 208 can be connected to a delivery site 207. The delivery site 207 is a site to which the fluid is to be delivered by the fluid management system 200. In some examples, the delivery site 207 comprises an anatomical structure (e.g., organ, body cavity) of a patient, and the fluid outlet 208 can be connected to a trocar that provides fluid communication with the anatomical structure of the patient. Accordingly, the fluid management system 200 can be used to deliver air (e.g., gas for insufflation) and/or liquid to a target delivery site.

The fluid management system 200 further comprises a third chamber 210. The third chamber 210 is connected to the first chamber 202 via a first set of one or more openings 212 and connected to the second chamber 206 via a second set of one or more openings 214. The third chamber can be pressurized at a desirable pressure setpoint. For example, the third chamber can enclose air that is pressurized at the desirable pressure setpoint.

The third chamber 210 can be pressurized using a pressure regulation device 216, which controls and maintains a desired pressure level within the third chamber 210. In some examples, the pressure regulation device 216 is an air pump that can control and maintain the air pressure within the third chamber 210 at the desirable pressure setpoint. In some examples, the air pump can increase the pressure of the air in the third chamber 210 to a pressure equaling the desired setpoint pressure minus a relatively small offset to account for the force required to open the flexible non-permeable membrane (e.g., up to 30 millimeters of mercury). In some examples, the pressure regulation device 216 comprises an air supply and another type of back-pressure regulator. In some examples, the pressure regulation device 216 comprises a mechanical regulator (e.g., a spring or pilot-based regulator).

The desirable pressure setpoint can refer to the pressure at which the fluid is delivered to the patient (i.e., the pressure of the fluid applied to the delivery site). The desirable pressure setpoint may be predetermined (e.g., based on the procedure, patient-specific information, or the like) or determined in situ (e.g., based on medical events during the procedure, equipment performance/tolerances, etc.). In some examples, the desirable pressure setpoint can be specified by a user via a software program associated with the pressure regulation device 216. In some examples, the user can specify a type of procedure (e.g., via a user interface for selecting procedure types, via bar codes, via RFID tags) and the software program can automatically determine a desirable pressure setpoint based on the specified type of procedure.

The fluid management system 200 further comprises a flexible membrane 240. The flexible membrane 240 is located in the third chamber 210 and is non-permeable. Thus, the flexible membrane 240 can fluidly isolate the third chamber 210 from both the first chamber 202 and the second chamber 206, meaning that any fluid in the first chamber 202 and the second chamber 206 cannot travel through the flexible membrane 240. As described below, the flexible membrane 240 is movable by a differential pressure between the third chamber 210 and the second chamber 214 such that the first chamber 212 and the second chamber 214 can be fluidly connected via the first set of one or more openings 212 and the second set of one or more openings 214 to regulate the pressure of the fluid delivered at the fluid outlet 208. In some examples, the flexible membrane 240 is configured such that the pressure in the third chamber 210 causes the flexible membrane 240 to stretch away from the wall 220, which in turn can cause the regulated vacuum pressure supplied to the surgical site to be between about 10 mmHg and about 30 mmHg greater than the pressure provided by the pressure regulation device 216 (e.g., due to the offset to account for the force required to displace the flexible non-permeable membrane 240). The flexible membrane 240 can be made of, for example, neoprene, silicone, natural rubber, nitrile, EPDM, other rubber compounds, or any other material that allows the flexible membrane to be displaced via pressure.

FIG. 2A illustrates exemplary operations of the fluid management system when the pressure in the third chamber 210 (i.e., the desirable pressure setpoint) equals or is higher than the pressure in the second chamber 206, in accordance with some examples. As shown in FIG. 2A, the higher pressure in the third chamber 210 pushes the flexible membrane 210 against the wall 220, thus sealing the first set of openings 212 and the second set of openings 214 on the wall 220. Thus, any fluid in the first chamber 202 and the second chamber 206 is blocked from traveling through the first set of openings 212 and the second set of openings 214. Instead, any fluid received via the fluid inlet 204 would enter the first chamber 202, travel through the flexible tube 230 to enter the second chamber 206, and exit the second chamber 206 via the fluid outlet 208. At the fluid outlet 208, the exiting fluid is delivered at the same pressure as the pressure in the second chamber 206, which equals or is lower than the pressure in the third chamber (i.e., the desirable pressure setpoint) 210.

FIG. 2B illustrates exemplary operations of the fluid management system when the pressure in the third chamber 210 (i.e., the desirable pressure setpoint) is lower than the pressure in the second chamber 214, in accordance with some examples. As shown in FIG. 2B, the higher pressure in the second chamber 206 pushes the flexible membrane 210 away from the wall 220, thus opening a fluid passage for fluid to travel through the second set of openings 214 into the third chamber 210 and then through the first set of openings 212 into the first chamber 202. Any excess fluid in the first chamber 202 is automatically reclaimed by the spinning of the rollers under the normal operation of the peristaltic pump. The circulation of fluid from the second chamber 206 back to the first chamber 202 can reduce the pressure in the second chamber 206, thus reducing the pressure of the fluid delivered to the delivery site 207 via outlet 208. The displacement of the flexible membrane 210 continues as long as the pressure in the second chamber 206 is higher than the pressure in the third chamber 210, thus causing the pressure in the second chamber 206 to continue to drop. When the pressure in the second chamber 206 becomes equal to the pressure in the third chamber (i.e., the desirable pressure setpoint) 210, the flexible membrane 240 returns to its original position shown in FIG. 2A to seal off the first set of openings 212 and the second set of openings 214 on the wall 220. The fluid management system 200 thus returns to the configuration in FIG. 2A, in which the fluid is delivered at the same pressure as the pressure in the second chamber 206, which equals or is lower than the pressure in the third chamber 210 (i.e., the desirable pressure setpoint).

In summary, the flexible membrane 240 can act as a back-pressure regulator valve between the first chamber 202 and the second chamber 206. The displacement of the flexible membrane 240 allows excess pressure in the second chamber 206 to be relieved via fluid flow from the second chamber 206 into the first chamber 202. Accordingly, the mechanism ensures that the fluid pressure at the fluid outlet 208 of the fluid management system 200 remains equal to or below the desirable pressure setpoint, regardless of variations in flow rate or upstream pressure. Specifically, the pump 218 of the fluid management system 200 can pull fluid at any rate (e.g., from fluid source 205) and the pressure at the fluid outlet 208 will remain consistently at or below the desired setpoint. The consistent pressure can be maintained as long as the fluid passage created between the first chamber 202 and the second chamber 206 via the displacement of the flexible membrane 240 is sized appropriately to allow the relief of pressure when the pump 218 is running at full speed.

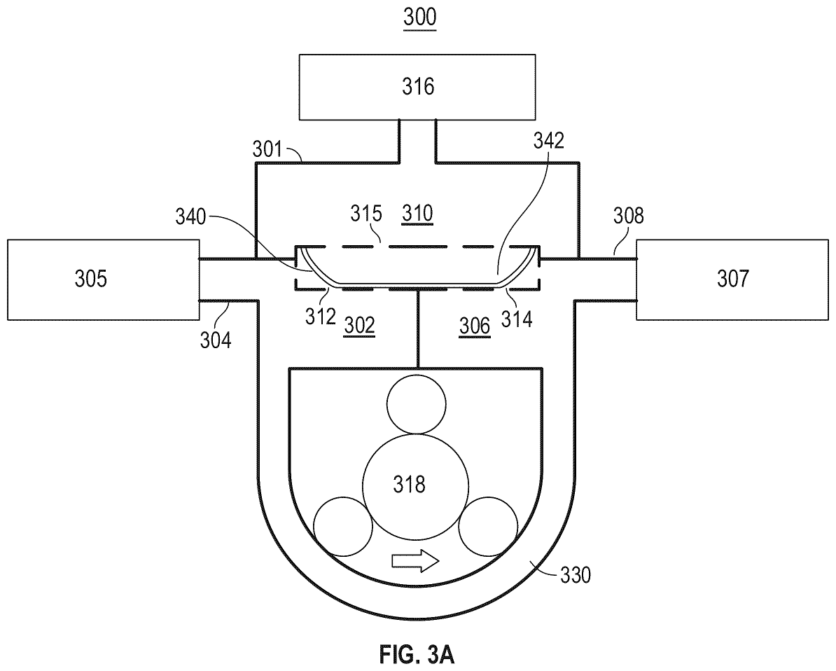

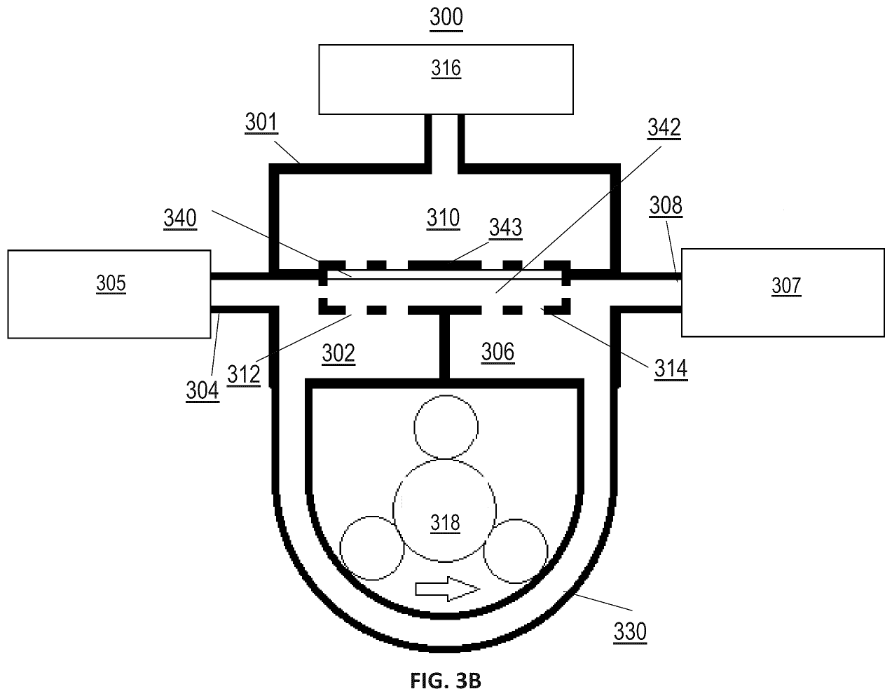

FIGS. 3A-B illustrate an exemplary fluid management system 300 for medical usage, in accordance with some examples. The fluid management system 300 comprises a body 301, which can be coupled to a fluid source 305, a delivery site 307, a pressure regulation device 316 (e.g., a software-controlled pump), and a pump 318. In some examples, the body 301 can be made of a disposable material. Accordingly, the body 301 can be detached from the other components and discarded after use (e.g., after a medical procedure) and the other components can be re-used with a new body (e.g., to manage a different type of fluid, to be used for a different patient, or to be used in a different medical procedure).

Similar to the fluid management system 200, the fluid management system 300 (specifically, the body 301) comprises a first chamber 302 having a fluid inlet 304 and a second chamber 306 having a fluid outlet 308. The fluid management system 300 further comprises a pump 318. The pump 318 is configured to transfer fluid received from the first chamber 302 to the second chamber 306. In some examples, the pump 318 is a peristaltic pump that can operate as described with reference to FIGS. 1A-C. Similar to the fluid management system 200, the fluid inlet 304 can be connected to a fluid source 305. The fluid source 305 can provide a variety of fluids, such as liquid (e.g., saline, a contrast agent, a medication, or the like) or air, to the fluid management system 300. The fluid outlet 308 can be connected to a delivery site 307. The delivery site 307 is a site to which the fluid is to be delivered by the fluid management system 300. Accordingly, the fluid management system 300 can be used to deliver gas (e.g., for insufflation) and/or liquid to a target delivery site.

The fluid management system 300 further comprises a third chamber 310. The third chamber can be pressurized at the desirable pressure setpoint. For example, the third chamber can enclose air that is pressurized at the desirable pressure setpoint. Similar to the fluid management system 200, the third chamber 310 can be pressurized via a pressure regulation device 316, which can operate similar to the pressure regulation device 216 described herein.

The third chamber 310 includes an intermediate chamber 342. As shown in FIGS. 3A-B, the intermediate chamber 342 is adjacent to the first chamber 302 and the second chamber 306. The third chamber 310 is connected to the first chamber 302 via the intermediate chamber 342, which has a first set of one or more openings 312, and is also connected to the second chamber 306 via the intermediate chamber 342, which has a second set of one or more openings 314.

The fluid management system 300 further comprises a flexible membrane 340. The flexible membrane 340 is located in the intermediate chamber 342 of the third chamber 310 and is non-permeable. Thus, the flexible membrane 340 can fluidly isolate the third chamber 310 from both the first chamber 302 and the second chamber 306, meaning that any fluid in the first chamber 302 and the second chamber 306 cannot travel through the flexible membrane 340. As described below, the flexible membrane 340 is movable by a differential pressure between the third chamber 310 and the second chamber 314 such that the first chamber 312 and the second chamber 314 can be fluidly connected via the first set of one or more openings 312 and the second set of one or more openings 314 to regulate the pressure of the fluid delivered at the fluid outlet 308. Further, the intermediate chamber 342 comprises one or more openings 315 at the top. The openings 315 at the top of the intermediate chamber 342 allow the air from the third chamber 310 into the intermediate chamber 342 to force the membrane 340 down to cover the first set of one or more openings 312 and the second set of one or more openings 314. Further, the top of the intermediate chamber with the openings 315 may serve as a barrier, or provide structural support, to keep the membrane 340 from overstretching as the fluid flows from the second chamber 314 to the first chamber 312 in an overpressure event.

FIG. 3A illustrates exemplary operations of the fluid management system 300 when the pressure in the third chamber 310 (i.e., the desirable pressure setpoint) equals or is higher than the pressure in the second chamber 306, in accordance with some examples. As shown in FIG. 3A, the higher pressure in the third chamber 310 pushes the flexible membrane 310 against the lower wall of the intermediate chamber 342, thus sealing the first set of openings 312 and the second set of openings 314. Thus, any fluid in the first chamber 302 and the second chamber 306 are blocked from traveling through the first set of openings 312 and the second set of openings 314. Instead, any fluid received via the fluid inlet 304 would enter the first chamber 302, travel through the flexible tube 330 to enter the second chamber 306, and exit the second chamber 306 via the fluid outlet 308. At the fluid outlet 308, the exiting fluid is delivered at the same pressure as the pressure in the second chamber 306, which equals or is lower than the pressure in the third chamber 310 (i.e., the desirable pressure setpoint).

FIG. 3B illustrates exemplary operations of the fluid management system 300 when the pressure in the third chamber 310 (i.e., the desirable pressure setpoint) is lower than the pressure in the second chamber 306, in accordance with some examples. As shown in FIG. 3B, the higher pressure in the second chamber 306 pushes the flexible membrane 310 against the top wall 343 of the intermediate chamber 342, thus opening a fluid passage for fluid to travel through the second set of openings 314 into the third chamber 310 and then through the first set of openings 312 into the first chamber 302. The circulation of fluid from the second chamber 306 back to the first chamber 302 can reduce the pressure in the second chamber 306. The opening of the fluid path in the intermediate chamber 341 will continue as long as the pressure in the second chamber 306 is higher than the pressure in the third chamber 310, thus causing the pressure in the second chamber 306 to continue to drop. When the pressure in the second chamber 306 becomes equal to or lower than the pressure in the third chamber (i.e., the desirable pressure setpoint) 310, the flexible membrane 340 returns to the displaced position shown in FIG. 3A to seal off the first set of openings 312 and the second set of openings 314. The fluid management system 300 thus returns to the configuration in FIG. 3A, in which the fluid is delivered at the same pressure as the pressure in the second chamber 306, which equals or is lower than the pressure in the third chamber (i.e., the desirable pressure setpoint) 310.

The fluid management systems 200 and 300 have various advantages. The fluid management system 200 may be cheaper to manufacture than the fluid management 300 as the fluid management system 200 involves fewer molded components. The fluid management system 300 may provide a safer default mode of operation than the fluid management system 200. For example, if fluid management system 300 loses the ability to regulate pressure in the third chamber 310 and the pressure in the third chamber 310 becomes negligible, the flexible membrane 342 is pushed against the top wall 343 of the intermediate chamber 342 to open the fluid path between the first chamber 302 and the second chamber 306. This way, the pump 318 may stop drawing new fluid from the fluid source 305 and the existing fluid in the fluid management system 300 will be circulated between the first chamber 302 and the second chamber 306 continuously, thus preventing excessive pressure from building up at the fluid outlet 308.

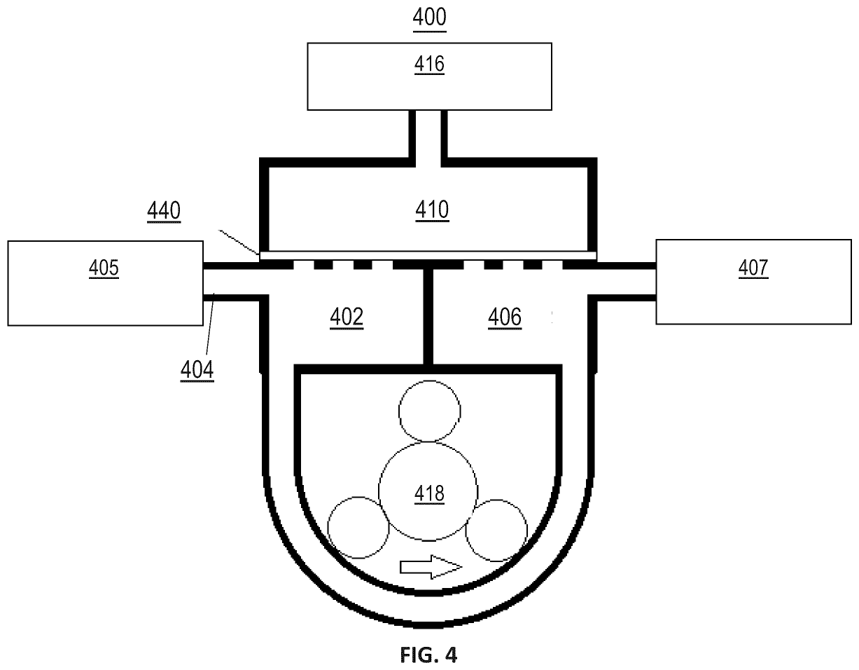

In FIGS. 2A-B and 3A-B, the body 201 (of fluid management system 300) and the body 301 (of fluid management system 300) are configured to deliver fluid from a fluid inlet to a fluid outlet. Without altering their construction, either the body 201 or the body 301 can be configured to suction fluids (e.g., liquid, air) away from a fluid inlet toward a fluid outlet. FIG. 4 illustrates an exemplary fluid management system 400, in accordance with some examples. In FIG. 4, the body 401 may be identical to the body 201 in FIGS. 2A-B. The body 401 is connected to a pressure regulation device 416 (e.g., a software-controlled pump). In some examples, the pressure regulation device 416 can comprise a vacuum pump, which removes air or gas molecules from the third chamber 410 to create a vacuum in the third chamber 410. By the same principles of operation, the pressure in the second chamber 406 is maintained at zero or negative, thus creating a suction force. In some examples, the pressure regulation device 416 can operate as described above with reference to the pressure regulation device 216 in FIGS. 2A-2B to pressurize the third chamber 410 at a pressure setpoint while the vacuum is created by the pump 418 pulling in air through the fluid inlet 404.

The body 401 is connected to a vacuum site 405 via the fluid inlet 404. The vacuum site 405 may be a location in/on a patient (e.g., cavities, passages, surgical sites, wounds) where suction is needed. For example, in endoscopy procedures, suction can be used to remove secretions, blood, or debris from the gastrointestinal or respiratory tract to enhance visualization. As another example, during dental procedures or oral surgeries, suction can be used to remove saliva, blood, and debris from the mouth to improve visibility and prevent aspiration. The body 401 is further connected to a waste collection site 407 (e.g., a drain) for collecting the suctioned waste.

The configuration of the fluid management system 400 can provide consistent regulation while running the pump at full speed to produce maximum vacuum without the need to modulate the pumps speed. In some examples, multiple fluid management systems (e.g., 200, 300, 400) as described herein can be used together in a single medical procedure to provide delivery and/or suction of various fluids as needed.

Any of the fluid management systems described herein can provide a mechanism for automatically adjusting the speed of the fluid pump (e.g., pump 218, pump 318, pump 418). In some examples, the fluid management system can include a sensor (e.g., a motion sensor, piezo sensor, and the like) for detecting displacement of the flexible membrane. In some examples, the displacement of the flexible membrane can be detected via a sudden drop in pressure in the second chamber. Additionally or alternatively, the displacement of the flexible membrane can be detected by a software module on the pressure regulation device (e.g., device 216, 316, 416). For example, the software module may detect a relatively small increase in pressure inside the third chamber, which can be caused by the displaced membrane decreasing the volume of the third chamber momentarily. The displacement of the flexible membrane is an indication of excess pressure in the second chamber. Thus, if the displacement of the flexible membrane is detected, the speed of the fluid pump can be automatically reduced to help relieve the excess pressure in the second chamber.

Further, if the displacement persists for a prolonged period of time, it may indicate that no new fluid is drawn at the fluid inlet and the fluid pump is circulating the same fluid between the first chamber and the second chamber (e.g., because no fluid is dispensed at the fluid outlet due to obstructions). Thus, if the displacement of the flexible membrane is detected to exceed a threshold time period (e.g., 3-5 seconds), the fluid pump can be stopped. Any aspects of the operations of the fluid management systems described herein, such as the displacement of the flexible membrane at various times and the pressure at the fluid inlet/fluid outlet at various times, can be monitored, recorded, and/or reported to a user.

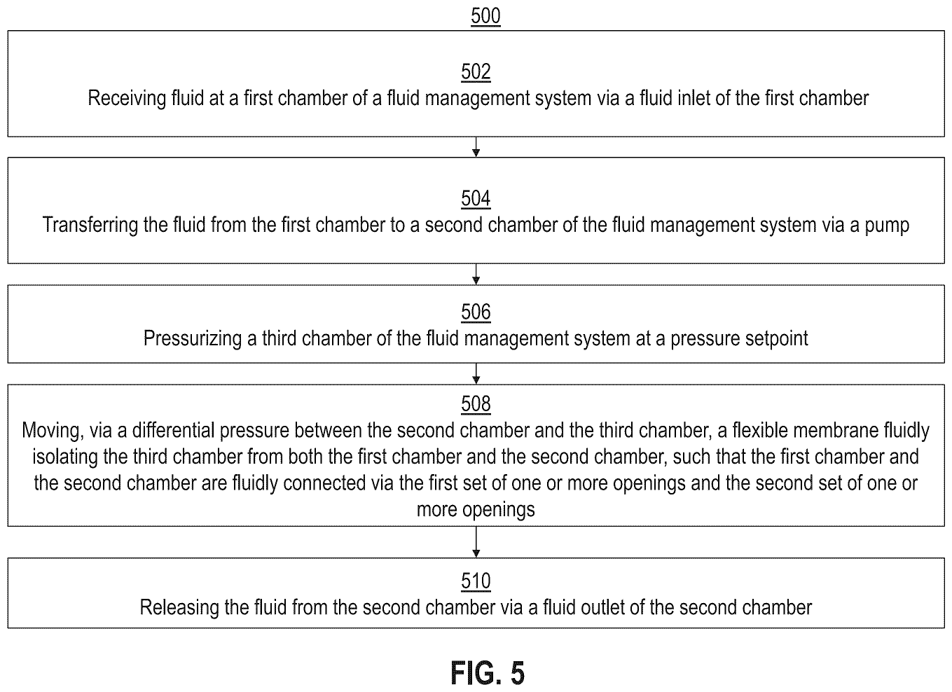

FIG. 5 illustrates an exemplary method 500 for providing fluid management, in accordance withs some examples. Process 500 is performed, for example, using any of the fluid management systems described herein. In process 500, some blocks are, optionally, combined, the order of some blocks is, optionally, changed, and some blocks are, optionally, omitted. In some examples, additional steps may be performed in combination with the process 500. Accordingly, the operations as illustrated (and described in greater detail below) are exemplary by nature and, as such, should not be viewed as limiting.

At block 502, a fluid management system receives fluid at a first chamber of a fluid management system via a fluid inlet of the first chamber. At block 504, the fluid management system transfers the fluid from the first chamber to a second chamber of the fluid management system via a pump. At block 506, the fluid management system pressurizes a third chamber of the fluid management system at a pressure setpoint. The third chamber is connected to the first chamber via a first set of one or more openings and connected to the second chamber via a second set of one or more openings. At block 508, the fluid management system moves, via a differential pressure between the second chamber and the third chamber, a flexible membrane fluidly isolating the third chamber from both the first chamber and the second chamber, such that the first chamber and the second chamber are fluidly connected via the first set of one or more openings and the second set of one or more openings. At block 510, the fluid management system releases the fluid from the second chamber via a fluid outlet of the second chamber.

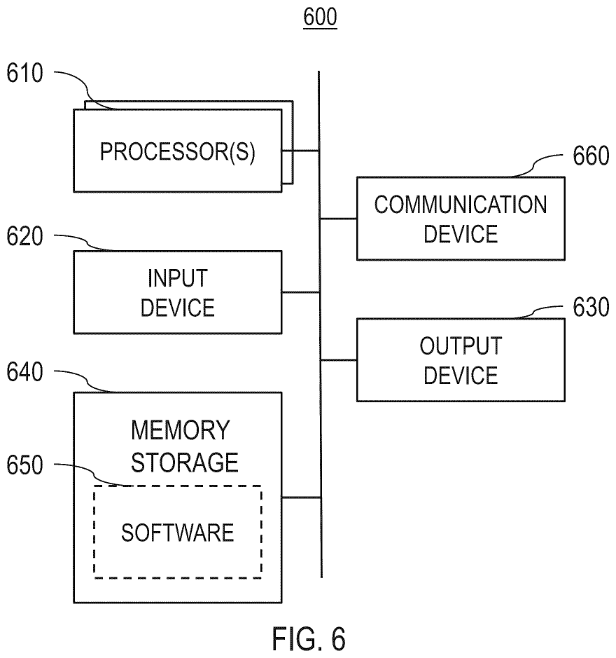

FIG. 6 illustrates an example of a computing device in accordance with one embodiment. Device 600 can be a host computer connected to a network. Device 600 can be a client computer or a server. As shown in FIG. 1, device 600 can be any suitable type of microprocessor-based device, such as a personal computer, workstation, server or handheld computing device (portable electronic device) such as a phone or tablet. The device can include, for example, one or more of processor 610, input device 620, output device 630, storage 640, and communication device 660. Input device 620 and output device 630 can generally correspond to those described above, and can either be connectable or integrated with the computer.

Input device 620 can be any suitable device that provides input, such as a touch screen, keyboard or keypad, mouse, or voice-recognition device. Output device 630 can be any suitable device that provides output, such as a touch screen, haptics device, or speaker.

Storage 640 can be any suitable device that provides storage, such as an electrical, magnetic or optical memory including a RAM, cache, hard drive, or removable storage disk. Communication device 660 can include any suitable device capable of transmitting and receiving signals over a network, such as a network interface chip or device. The components of the computer can be connected in any suitable manner, such as via a physical bus or wirelessly.

Software 650, which can be stored in storage 640 and executed by processor 610, can include, for example, the programming that embodies the functionality of the present disclosure (e.g., as embodied in the devices as described above).

Software 650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 640, that can contain or store programming for use by or in connection with an instruction execution system, apparatus, or device.

Software 650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 600 can implement any operating system suitable for operating on the network. Software 650 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

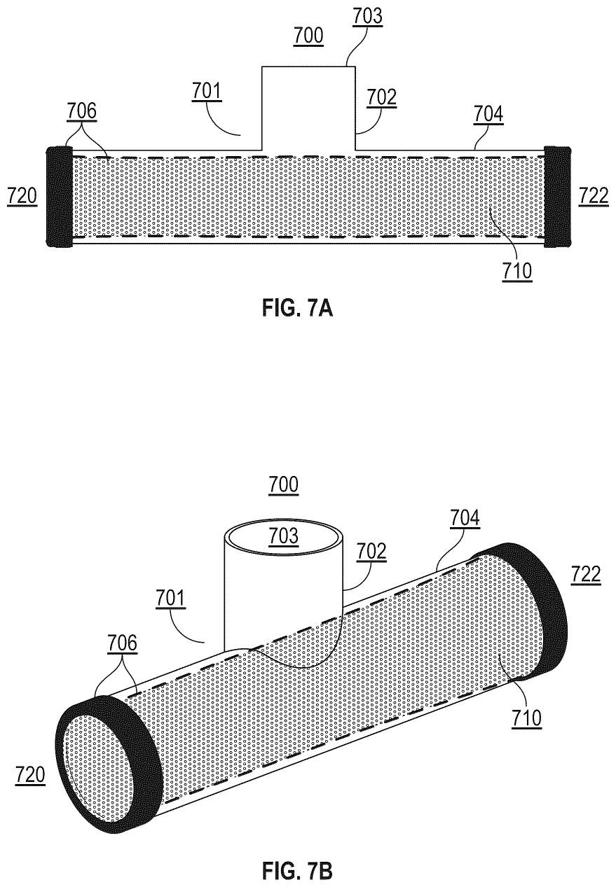

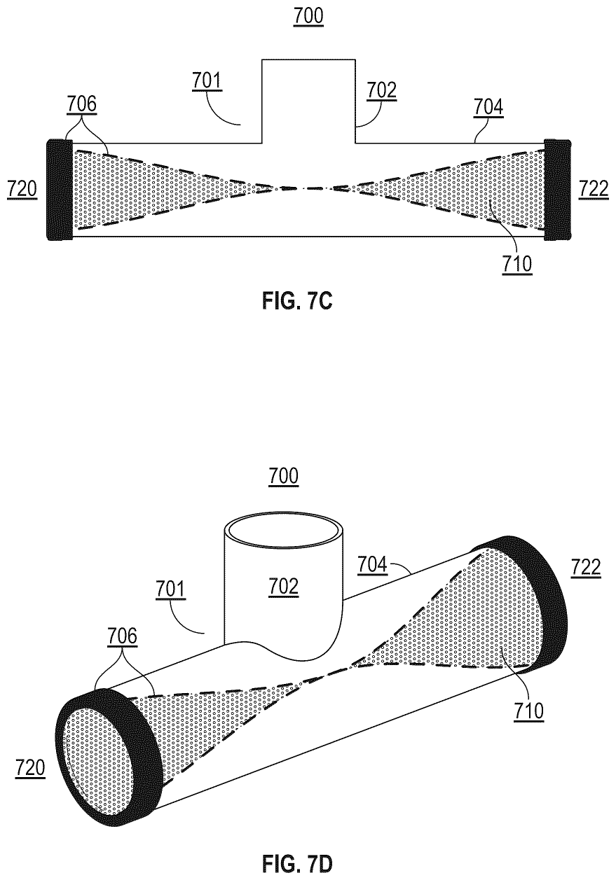

FIGS. 7A-D illustrate an exemplary fluid management system 700 for medical usage, in accordance with some examples. The fluid management system 700 comprises a body 701, which includes two rigid tubular segments 702 and 704. The two rigid tubular segments 702 and 704 are connected to each other. In some examples, the two rigid tubular segments 702 and 704 may comprise a single body (e.g., via mold injecting). In the depicted example, the bottom end of the tubular segment 702 is connected to a side surface of the tubular segment 704. The tubular segment 702 and the tubular segment 704 are fluidly connected, such that fluid (e.g., air) can travel between the tubular segment 702 and the tubular segment 704.

With reference to FIG. 7A, the tubular segment 704 has two open ends 720 and 722. One of the two open ends (e.g., open end 720) can be coupled to a fluid source. The fluid source can provide a variety of fluids, such as liquid (e.g., saline, a contrast agent, a medication, or the like) or air. The other open end (e.g., open end 722) can be coupled to a delivery site. The delivery site is a site to which the fluid is to be delivered by the fluid management system 700. The open end 703 of the tubular segment 702 can be coupled to a pressure regulation device (e.g., a software-controlled pump). In operation, the fluid management system 700 can be used to deliver gas (e.g., for insufflation) and/or liquid to a target delivery site. For example, the fluid can be delivered from the fluid source via the open end 720 of the tubular segment 704 to the delivery site via the open end 722 of the tubular segment 704, as described in detail below.

The fluid management system 700 further comprises a flexible lumen 706. The flexible lumen can be made of a sterile flexible material that can deform (e.g., stretch) upon application of a force (e.g., differential pressure) without breaking and can return to its original shape when the force is removed. Examples of the flexible material can include neoprene, silicone, natural rubber, nitrile, EPDM, other rubber compounds, or any other material that allows the flexible lumen 706 to be displaced via pressure. In some examples, the flexible lumen 706 may be comprised of, or include, the same material of the flexible membrane described herein (e.g., 240, 340). As shown in FIG. 7A, the flexible lumen 706 is threaded through the rigid tubular segment 704. The two ends of the flexible lumen 706 are affixed to the two ends 720 and 722 of the rigid tubular segment 704, respectively. In the depicted example, each end of the flexible lumen 706 is affixed to the tubular segment 704 by wrapping around a respective open end of the tubular segment 704. The flexible material of the flexible lumen 706 provides elasticity and friction such that each end of the lumen 706 is stretched to wrap around and cling to the respective open end of the tubular segment 704. Accordingly, when the top end 703 of the tubular segment 702 is connected to the pressure regulation device, a closed space is formed between the body 701 (as the outer layer) and the flexible lumen 706 (as the inner layer). In operation, the flexible lumen 706 can be stretched by a differential pressure to regulate the fluid flowing through the flexible lumen 706, as described in detail below.

In FIGS. 7A-B, one of the two open ends (e.g., open end 720) is coupled to a fluid source and the other open end (e.g., open end 722) is coupled to a delivery site. The fluid source outputs a fluid 710, which is delivered to the delivery site via the flexible lumen 706 within the rigid tubular segment 704. In some examples, a pump may be used upstream to control the output of the fluid 710 into the fluid management system 700. A pressure regulation device can be connected to the tubular segment 702 via the open end 703 and can set the pressure setpoint at the open end 703. The pressure regulation device can operate similarly to the pressure regulation device 216, 316, and/or 416 described herein. In some examples, the pressure regulation device is an air pump that can control and maintain the air pressure within the closed space between the body 701 (as the outer layer) and the flexible lumen 706 (as the inner layer). In some examples, the pressure regulation device comprises an air supply and another type of back-pressure regulator. In some examples, the pressure regulation device comprises a mechanical regulator (e.g., a spring or pilot-based regulator).

The fluid management system can determine whether the pressure at the delivery site is within an acceptable range. Importantly, different organs or anatomical structures may be associated with different acceptable pressure thresholds depending on factors such as tissue sensitivity, vascularization, and the type of procedure being performed. For example, the pressure at which fluids can be delivered to a bladder should generally not exceed 75 millimeters of mercury. In some examples, the pressure at the delivery site may be measured by one or more pressure sensors, which may be located near or at the delivery site. In some examples, the pressure at the delivery site can be determined using a sensor (e.g., a motion sensor, piezo sensor, and the like) that detects displacement of the flexible lumen 706. For example, the increased pressure at the delivery site may cause the flexible lumen 706 to expand outwardly and the outward expansion of the flexible lumen 706 can be detected by, for example, a software module on the pressure regulation device. For example, the software module may detect a relatively small increase in pressure inside the closed space, which can be caused by the outwardly expanding flexible lumen decreasing the volume of the closed space momentarily. The outward expansion of the flexible lumen 706 is an indication of excess pressure within the flexible lumen 706 (and thus the delivery site). The amount of expansion of the flexible lumen 706 can be used to determine whether the pressure at the delivery site is within an acceptable range.

In FIGS. 7A-B, the fluid management system 700 can determine that the pressure at the delivery site is within an acceptable range. In accordance with a determination that the pressure is within an acceptable range, the pressure regulation device connected to the tubular segment 702 does not increase pressure to reduce the flow of the fluid 710.

In FIGS. 7C-D, the pressure at the delivery site exceeds the acceptable range. The pressure at the delivery site may be measured by one or more pressure sensors, which may be located near or at the delivery site. In accordance with a determination that the pressure exceeds the acceptable range, the pressure regulation device connected to the tubular segment 702 can increase the pressure in the closed space formed between the flexible lumen 706 and the body 701. The increased pressure can cause the flexible lumen 706 to stretch inwardly, thereby restricting the flow through the flexible lumen 706 to relieve the pressure at the delivery site. The pressure in the closed space between the body 701 (as the outer layer) and the flexible lumen 706 (as the inner layer) may be increased enough such that the flexible lumen 706 completely cuts off the flow of the fluid as shown in FIGS. 7C-D. Additionally or alternatively, the speed of the fluid pump can be automatically reduced to help relieve the excess pressure at the delivery site. With the flexible lumen 706 momentarily reducing or restricting flow through the flexible lumen 706, the fluid management system 700 may determine that the pressure at the delivery site no longer exceeds the acceptable range and the pressure regulation device can decrease the pressure in the closed space formed between the flexible lumen 706 and the body 701. Thus, the configuration of the fluid management system 700 can provide consistent regulation while running the pump at full speed without the need to modulate the pump speed.

In the depicted examples in FIGS. 7A-D, the tubular segments 702 and 704 are of cylindrical shape. But it should be appreciated that each tubular segment may be of other shapes, such as a rectangular prism, a cube, or an irregular shape. Without altering the construction, the fluid management system 700 can be configured to suction fluids (e.g., liquid, air) away from a fluid inlet toward a fluid outlet.

The examples in FIGS. 7A-D provide several advantages. The body 701 formed by the tubular segments 702 and 704 can be easier to manufacture because it can be relatively small in size, does not contain multiple rigid internal chambers, and can be made using readily available rigid materials. In some examples, the body 701 can be made of a disposable material. Accordingly, the body 701 can be detached from the other components and discarded after use (e.g., after a medical procedure) and the other components can be re-used with a new body (e.g., to manage a different type of fluid, to be used for a different patient and/or in a different medical procedure).

The foregoing description, for the purpose of explanation, has been described with reference to specific examples or aspects. However, the illustrative discussions above are not intended to be exhaustive or to limit the invention to the precise forms disclosed. For the purpose of clarity and a concise description, features are described herein as part of the same or separate variations; however, it will be appreciated that the scope of the disclosure includes variations having combinations of all or some of the features described. Many modifications and variations are possible in view of the above teachings. The variations were chosen and described in order to best explain the principles of the techniques and their practical applications. Others skilled in the art are thereby enabled to best utilize the techniques and various variations with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

What is claimed is:

1. A fluid management system for medical usage, comprising:
a first chamber having a fluid inlet;
a second chamber having a fluid outlet;
a third chamber configured to be pressurized at a pressure setpoint, wherein the third chamber is connected to the first chamber via a first set of one or more openings and connected to the second chamber via a second set of one or more openings;
a pump configured to transfer fluid received from the first chamber to the second chamber; and
a flexible membrane fluidly isolating the third chamber from both the first chamber and the second chamber, wherein the flexible membrane is movable by a differential pressure between the third chamber and the second chamber such that the first chamber and the second chamber are fluidly connected via the first set of one or more openings and the second set of one or more openings.

2. The fluid management system of claim 1,
wherein the fluid transferred by the pump comprises liquid and/or air, and
wherein the fluid management system is configured to receive the fluid from the fluid source and provide the fluid via the fluid outlet at or below the pressure setpoint.

3. The fluid management system of claim 2, wherein the third chamber is configured to be pressurized via air.

4. The fluid management system of claim 2, wherein the liquid comprises saline, a contrast agent, a medication, or any combination thereof.

5. The fluid management system of claim 2, wherein the fluid source comprises one or more intravenous (IV) containers and the fluid outlet is connected to a patient.

6. The fluid management system of claim 1,
wherein the fluid transferred by the pump comprises liquid and/or air, and
wherein the fluid management system is configured to receive the fluid from a surgical site and provide the fluid via the fluid outlet at or below the pressure setpoint.

7. The fluid management system of claim 6, wherein the third chamber is a vacuum chamber.

8. The fluid management system of claim 6, wherein the fluid outlet is connected to a waste drain.

9. The fluid management system of claim 1, wherein the pump comprises a peristaltic pump.

10. The fluid management system of claim 1, wherein the pressure setpoint in the third chamber is based on a user input.

11. The fluid management system of claim 10, wherein the user input is received by a software-controlled pump configured to regulate pressure in the third chamber.

12. The fluid management system of claim 1, wherein the third chamber comprises an intermediate chamber adjacent to the first chamber and the second chamber, and wherein the flexible membrane is located within the intermediate chamber.

13. The fluid management system of claim 1, further comprising a sensor for detecting displacement of the flexible membrane.

14. The fluid management system of claim 13, wherein the sensor comprises a motion sensor.

15. The fluid management system of claim 13, wherein the fluid management system is configured to reduce a speed of the pump if the displacement of the flexible membrane is detected.

16. The fluid management system of claim 13, wherein the fluid management system is configured to stop the pump if the displacement of the flexible membrane exceeds a threshold time period.

17. A method for providing fluid management, comprising:
receiving fluid at a first chamber of a fluid management system via a fluid inlet of the first chamber;
transferring the fluid from the first chamber to a second chamber of the fluid management system via a pump;
pressurizing a third chamber of the fluid management system at a pressure setpoint, wherein the third chamber is connected to the first chamber via a first set of one or more openings and connected to the second chamber via a second set of one or more openings;
moving, via a differential pressure between the second chamber and the third chamber, a flexible membrane fluidly isolating the third chamber from both the first chamber and the second chamber, such that the first chamber and the second chamber are fluidly connected via the first set of one or more openings and the second set of one or more openings; and
releasing the fluid from the second chamber via a fluid outlet of the second chamber.

18. The method of claim 17, wherein the fluid transferred by the pump comprises liquid and/or air, and
wherein the fluid management system is configured to receive the fluid from the fluid source and provide the fluid via the fluid outlet at or below the pressure setpoint.

19. A fluid management system configured to be coupled to a pump for medical usage, comprising:
a first chamber having a fluid inlet, wherein the first chamber is configured to obtain fluid via the fluid inlet;
a second chamber having a fluid outlet, wherein the second chamber is configured to receive the fluid via the pump and release the fluid via the fluid outlet;
a third chamber configured to be pressurized at a pressure setpoint, wherein the third chamber is connected to the first chamber via a first set of one or more openings and connected to the second chamber via a second set of one or more openings; and
a flexible membrane fluidly isolating the third chamber from both the first chamber and the second chamber, wherein the flexible membrane is movable by a differential pressure between the third chamber and the second chamber such that the first chamber and the second chamber are fluidly connected via the first set of one or more openings and the second set of one or more openings.

20. The fluid management system of claim 19, wherein the fluid transferred by the pump comprises liquid and/or air, and wherein the fluid management system is configured to receive the fluid from the fluid source and provide the fluid via the fluid outlet at or below the pressure setpoint.
